(19) United States
(12) Patent Application Publication
(10) Pub. No.: US 2012/0271142 A1
(43) Pub. Date: Oct. 25, 2012

(54) Apparatus for Migrating Anatomic Parts and Method of Using the Apparatus

(75) Inventor: Michael Campagna, Oak Park, IL (US)

(73) Assignee: DESIGN MD LLC., LIBERTYVILLE, IL (US)

(21) Appl. No.: 13/174,652

(22) Filed: Jun. 30, 2011

Related U.S. Application Data
(60) Provisional application No. 61/421,553, filed on Dec. 9, 2010.

(51) Int. Cl. A61F 5/37; A61B 6/00

(52) U.S. Cl. 600/407

(57) ABSTRACT

An apparatus comprises a first anatomic positioner for migrating a first anatomic part of a patient on a patient platform. The first anatomic positioner comprises an arch structure being configured to be operable for engaging the patient's shoulder area. A strap structure is joined to the arch structure to extend caudally along the patient's side. A locking system is configured to be operable for longitudinal movement along a side of the patient platform and for being locked at a position along the movement. The locking system is further configured for engaging the strap structure for pulling the arch structure to caudally migrate the first anatomic part. Another apparatus further comprises a second anatomic positioner for migrating a second anatomic part of a patient on a patient platform, the second anatomic positioner comprising a mirror image of the first anatomic positioner.

### CROSS-REFERENCE TO RELATED APPLICATIONS

The present Utility patent application claims priority benefit of the U.S. provisional applications for patent Ser. No. 61/421,553 entitled “A Low Profile, Non Metallic Imaging Compatible Method for Intra-Operative Radiographic Visualization of The Cervical Vertebrae”, filed on 09 Dec. 2010, and patent Ser. No. 61/098,757 entitled “Universal Table Mount for the Citow Cervical Visualizer”, filed on 20 Sep. 2008 under 35 U.S.C. 119(e).

The present Utility patent application also claims priority benefit under 35 U.S.C. 120 of Utility patent application Ser. No. 12/464,456 entitled “An Apparatus for Mounting an Anatomical Positioner on a Patient Care Platform”, filed on 12 May 2009 and U.S. Continuation-in-part patent application Ser. No. 12/684,934 entitled “Apparatus and Method for Radiolucent Anatomic Positioning” filed on 09 Jan. 2010 under 35 USC 111(a). The contents of these related provisional and patent applications are incorporated herein by reference for all purposes.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to medical equipment. More particularly, the invention relates to means for variable radiolucent anatomic positioning that may be used with many types of imaging technology including, but not limited to, Magnetic Resonance Imagers (MRI), Computer Assisted Tomagraphy (CAT Scan) and 3D Cone Beam Tomagraphy (O-Arm technology).

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Medical procedures involving the cervical spine, particularly surgery, require correct visualization of the vertebrae within the imaging array. Typically, a subject's shoulders obscure the lateral imaging of the cervical vertebrae. It is therefore an objective of the present invention to provide means for positioning the subject's shoulders during imaging that migrate the shoulders of the subject out of the line of sight of the lateral image of the cervical vertebrae.

Technologic advances in the field of surgical and diagnostic imaging are taking place which preclude the usage of metallic implements within the imaging array. As such, many technologies which currently utilize metal components are now unworkable within the magnetic array, as even components constructed from non ferromagnetic metals such as titanium, aluminum, and stainless steel, even though such metals pose no projectile danger within the environment of the magnetic resonance imaging array, nonetheless, as high attenuation objects within this field, cause, significant interference with, and render useless the data obtained thru the introduction of streak artifact . Additionally, the restrictive working bore size of emerging imaging technologies require the innovation of low profile solutions to current equipment design. Examples of this emerging type of imaging technology are MRI, CT and the newly developed O-Arm, a 3-D fluoroscopic array utilizing Computer Assisted Cone Beam Tomagraphy.

Although all of the aforementioned imaging technologies utilize fundamentally different approaches to achieve similar results, they nonetheless have similar restrictions. These devices utilize a restrictively narrow bore opening. Also, none of these devices is able to function properly with devices that incorporate the use of metal. Accordingly, it would be desirable and useful to provide positioning equipment to be used in conjunction with these imaging devices that fit into a narrow bore opening and do not comprise metallic components.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that traditional means of shoulder migration exist to solve the problem of intra-operative shoulder migration for purposes of improved lateral radiography of the cervical vertebral structures, with varying degrees of success and risk attendant to usage. One traditional method of migrating the subject's shoulders involves wrapping straps or Kurlix® bandages around the forearms or wrists of a subject and pulling forcefully upon these straps or bandages during imaging. However, this means of pulling on the arms or wrists with straps or Kurlix® bandages oftentimes leads to brachial plexus insult and injury and often delivers poor results, as well as subjecting surgical and clinical staff to unwanted risk thru proximity to the various imaging arrays. The risk of patient injury with this method is ever-present whether said traction is delivered via someone directly pulling on a wrist strap during imaging or via the use of a mechanical version of someone puling on a wrist strap such, as, but not limited to, a weight or a friction lock, which are provided in some prior art methods. In fact, a mechanical pulling means may aggravate this risk in that no practical means for variable tensioning of the migratory pressure is provided in the mechanical means. At present, such solutions rely upon the attachment of sand bags, weights, or crude means of fixating straps via pulling on the arms or wrists has the effect of transferring direct force to the soft tissues and delicate structures of the shoulder capsule, with less than efficient migration of the shoulders.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that another traditional means of migrating a subject's shoulders involves taping down the shoulders or migrating the trapezius muscles with or without a cotton harness for the entirety of the imaging procedure. This means oftentimes has the effect of causing brachial palsy, as neither taping nor usage of various harness systems provide a means of varying the position of the user during the procedure, yet merely position and hold the shoulders into an unalleviated and unnatural position for the entire length of the procedure. This may increase the risk of nerve damage, while concurrently aggravating the results thru restricted blood flow to the trapezius and structures of the shoulders, whether by direct taping or by a sand bag tied to a harness. Additionally, via spreading the motive force of distal migratory tension across the entire soft tissue of the shoulder, the amount of migration of the actual dense artifact causing structures within the imaging is ineffective since no concentration of positioning is effectively directed to the actual joint that obscures the lateral imaging. Furthermore, the application of distal migratory pressure across the entire shoulder and trapezius has the effect of migrating the entire patient, or at least causes the patient's position in relation to the surgeon in intra-operative applications to migrate, which can result in substantial risk in the usage of table mounted retractor systems of this type.

Neither of these traditional means utilizes rigid radiolucent positioning to migrate the acromionclavicular joint alone. Rather, these means either migrate the entire trapezius in a harness or pull on the arms or wrists thereby migrating the entire patient as opposed to the acromionclavicular joint. Thereby rendering little actual value in real usage as it is the structures of the acromionclavicular joint which typically occlude the proper lateral radiographic view of the cervical vertebral structures. Also, much of the prior art does not lock into place during use, thereby necessitating that staff members are exposed to cumulative radiographic tissue load with each usage as they hold the means in place. Additionally, prior art methods make no provisions for intra-operative variability of distal migration, the absence of which is clinically proven to lead to temporary and sometimes permanent brachial palsy deficit for the subject, for example, without limitation, loss of sensation in the hands, fingers and lower portions of the arm. Furthermore, many prior art methods require multiple operators for usage. As such, the traditional means of intra-operative distal migration of the shoulders are ill suited at best, and introduce an unacceptable level of risk. Some traditional prior art methods, for example without limitation a compression harness that holds down the trapezius muscles, involve a complex set up which may be incompatible with the present array of patient positioning platforms in current usage.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a side perspective view of an exemplary means for positioning the shoulders of a subject for improved lateral imaging of the cervical vertebral structures; and

FIG. 2 is a side perspective view of an exemplary shoulder positioning system for improved lateral imaging of the cervical vertebral structure of a subject, in accordance with an embodiment of the present invention.

Unless otherwise indicated illustrations in the figures are not necessarily drawn to scale.

### SUMMARY OF THE INVENTION

To achieve the forgoing and other objects and in accordance with the purpose of the invention, a variety of apparatus for migrating anatomic parts and methods of using the apparatus are described.

In one embodiment an apparatus comprises a first anatomic positioner for migrating a first anatomic part of a patient on a patient platform. The first anatomic positioner comprises an arch structure being configured to be operable for engaging the patient's shoulder area. A strap structure is joined to the arch structure to extend caudally along the patient's side. A locking system is configured to be operable for longitudinal movement along a side of the patient platform and for being locked at a position along the movement. The locking system is further configured for engaging the strap structure for pulling the arch structure to caudally migrate the first anatomic part. In another embodiment the arch is further operable for engaging the patient's acromioclavicular joint and caudally migrating the acromioclavicular joint. In yet another embodiment the locking system is further configured to be operated by an operator at a foot of the patient platform. In still another embodiment the arch structure comprises a radiolucent material. In another embodiment the arch structure is substantially rigid. In yet another embodiment the strap structure comprises a radiolucent material. In still another embodiment the strap structure further comprises separate ends for joining to legs of the first arch structure. In another embodiment the strap structure further comprises two straps for joining to legs of the first arch structure. In yet another embodiment the locking system further comprises a trigger mechanism for locking at the position. In still another embodiment the arch structure is padded. Another embodiment further comprises a second anatomic positioner for migrating a second anatomic part of a patient on a patient platform, the second anatomic positioner comprising a mirror image of the first anatomic positioner. Yet another embodiment further comprises a chest strap being joined between the strap structures of the first anatomic positioner and the second anatomic positioner to mitigate lateral migration of the arch structures. Still another embodiment further comprises a shoulder blade strap being joined between the strap structures of the first anatomic positioner and the second anatomic positioner to further mitigate lateral migration of the arch structures.

In another embodiment an apparatus comprises a first anatomic positioner for migrating a first acromioclavicular joint of a patient on a patient platform. The first anatomic positioner comprises means for engaging the patient's first acromioclavicular joint, means, being joined to the engaging means, for extending caudally along the patient's first side, and means, being joined to the extending means, for longitudinal movement along a first side of the patient platform, for pulling the engaging means to caudally migrate the first acromioclavicular joint, and for locking at a position along the movement where the first acromioclavicular joint has been migrated. The apparatus further comprises a second anatomic positioner for migrating a second acromioclavicular joint of a patient on a patient platform. The second anatomic positioner comprises means for engaging the patient's second acromioclavicular joint, means, being joined to the engaging means, for extending caudally along the patient's second side, and means, being joined to the extending means, for longitudinal movement along a second side of the patient platform, for pulling the engaging means to caudally migrate the second acromioclavicular joint, and for locking at a position along the movement where the second acromioclavicular joint has been migrated to provide a clear radiographic lateral imaging of the cervical vertebral structures of the patient. Another embodiment further comprises means for mitigating lateral migration of the engaging means.

In another embodiment an apparatus comprises a first anatomic positioner for migrating a first acromioclavicular joint of a patient on a patient platform. The first anatomic positioner comprises a substantially rigid arch being configured to be operable for engaging the patient's first acromioclavicular joint. The rigid arch comprises a radiolucent material. A strap structure is joined to legs of the rigid arch to extend caudally along the patient's first side. The strap structure comprises a radiolucent material. A locking system is joined to the strap structure. The locking system is configured to be operable for longitudinal movement along a first side of the patient platform for pulling the arch structure to caudally migrate the first acromioclavicular joint. The locking system further comprises a trigger mechanism for locking at a position along the movement where the first acromioclavicular joint has been migrated. The apparatus further comprises a second anatomic positioner for migrating a second acromioclavicular joint of a patient on a patient platform. The second anatomic positioner comprises a substantially rigid arch being configured to be operable for engaging the patient's second acromioclavicular joint. The rigid arch comprises a radiolucent material. A strap structure is joined to legs of the rigid arch to extend caudally along the patient's second side. The strap structure comprises a radiolucent material. A locking system is joined to the strap structure. The locking system is configured to be operable for longitudinal movement along a second side of the patient platform for pulling the arch structure to caudally migrate the second acromioclavicular joint. The locking system further comprises a trigger mechanism for locking at a position along the movement where the second acromioclavicular joint has been migrated to provide a clear radiographic lateral imaging of the cervical vertebral structures of the patient. Another embodiment further comprises a chest strap being joined between the strap structures to mitigate lateral migration of the arch structures. Yet another embodiment further comprises a shoulder blade strap being joined between the strap structures to further mitigate lateral migration of the arch structures. In still another embodiment the locking systems are further configured to be operated by an operator at a foot of the patient platform. In another embodiment the strap structures each further comprises two straps for joining to the legs of the arch structures. In yet another embodiment the arch structures are padded.

In another embodiment a method of using the apparatus comprising steps of joining the locking system of the first anatomic positioner to the side of the patient platform proximate the foot of the patient platform where the locking system is operable to move longitudinally along the side. The method further comprises the step of joining the strap structure, joined to the arch structure, to the locking system. The method further comprises the step of placing the arch structure on the shoulder area of the patient. The method further comprises the step of pulling on the locking system to caudally migrate the first anatomic part. The method further comprises the step of locking the locking system at the position when the first anatomic part has been migrated. In another embodiment the method further comprises the step of placing the arch structure on the shoulder area of the patient above the acromioclavicular joint. In yet another embodiment the method further comprises the step of operating the locking system from the foot of the patient platform. In still another embodiment the method further comprises the step of operating a trigger mechanism for locking the locking system. In another embodiment the method further comprises the steps of joining a locking system of a second anatomic positioner to a second side of the patient platform proximate the foot of the patient platform where the locking system is operable to move longitudinally along the second side. The method further comprises the step of joining a strap structure of the second anatomic positioner, joined to an arch structure of the second anatomic positioner, to the locking system. The method further comprises the step of placing the arch structure of the second anatomic positioner on a second shoulder area of the patient. The method further comprises the step of pulling on the locking system of the second anatomic positioner to caudally migrate a second anatomic part. The method further comprises the step of locking the locking system of the second anatomic positioner at a position when the second anatomic part has been migrated. Another embodiment further comprises the step of placing the arch structure of the second anatomic positioner on the second shoulder area of the patient above a second acromioclavicular joint. Yet another embodiment further comprises the step of operating the locking system of the second anatomic positioner from the foot of the patient platform. Still another embodiment further comprises the step of operating a trigger mechanism of the second anatomic positioner for locking the locking system of the second anatomic positioner. Another embodiment further comprises the step of joining a chest strap, on top of the patient's chest, between the strap structures of the first anatomic positioner and the second anatomic positioner to mitigate lateral migration of the arch structures. Yet another embodiment further comprises the step of joining a shoulder blade strap, beneath the patient's shoulder blades, between the strap structures of the first anatomic positioner and the second anatomic positioner to further mitigate lateral migration of the arch structures.

Other features, advantages, and objects of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

An embodiment of the present invention and at least one variation thereof provide a table mounted shoulder positioning system that delivers temporary, variable and rigid radiolucent positioning directly to a subject's acromionclavicular joint via pulling a portion of the system towards the subject's feet. Many embodiments are implemented for use within the confines of the bore of an O-Arm, portable magnetic imaging array, MRI, or other imaging technologies.

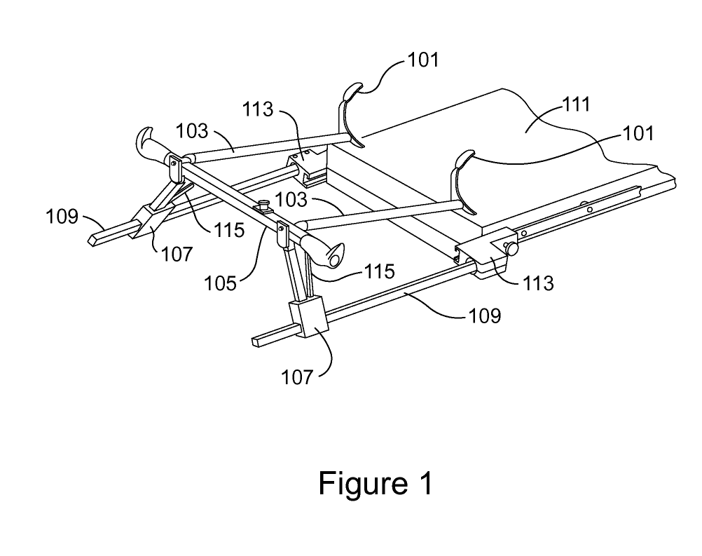

FIG. 1 is a side perspective view of an exemplary means for positioning the shoulders of a subject for improved lateral imaging of the cervical vertebral structures. In the present embodiment, offset arches 101 on the ends of pusher tubes 103 are used with a crossbar 105 to create a shoulder press to migrate the shoulders distally to allow for improved radiographic lateral views during cervical vertebral surgical and diagnostic procedures. Offset arches 101 and pusher tubes 103 are preferably constructed of radiographically invisible material, in order to deliver a platform for distal migration of the subject's acromionclavicular joint that does not appear on radiographic imaging. In some embodiments this pusher tube and arch assembly may be replaced by a one-piece arm made of high strength laminar sheeting that terminates in a variable geometric arch. In the present embodiment, the shoulder press is utilized as a component of a table mounted system. In this system, crossbar 105 is adjustable and is cradled into variably positionable table mounted rail guides 107 with an automatic hands free locking system. Rail guides 107 slide along rails 109 that are mounted to a table 111 by mounting means 113. Triggers 115 control the locking system by enabling rail guides 107 to slide along rails 109 when engaged by a user and locking rail guides 107 in place when released by the user. Therefore, shoulder press 105 may be positioned on a subject for proper imaging and held in place by the locking system without the aid of the user, such that nobody save the patient is subjected to radiation exposure during imaging.

This embodiment positions arches 101 directly atop the subject's acromionclavicular joint, such that the joint alone migrates distally rather than the entire subject migrating distally, and then releases all pressure as soon as the radiographic imagery is completed, thereby limiting distal migration to mere minutes as opposed to hours. This table mounted positioning system provides temporary distal migratory pressure to the structures of the acromionclavicular joint and delivers improved lateral radiographic images of the cervical vertebral structures in comparison to traditional means, with little incidence of the concomitant risks of brachial plexus insult or injury and palsy that are commonplace with traditional means.

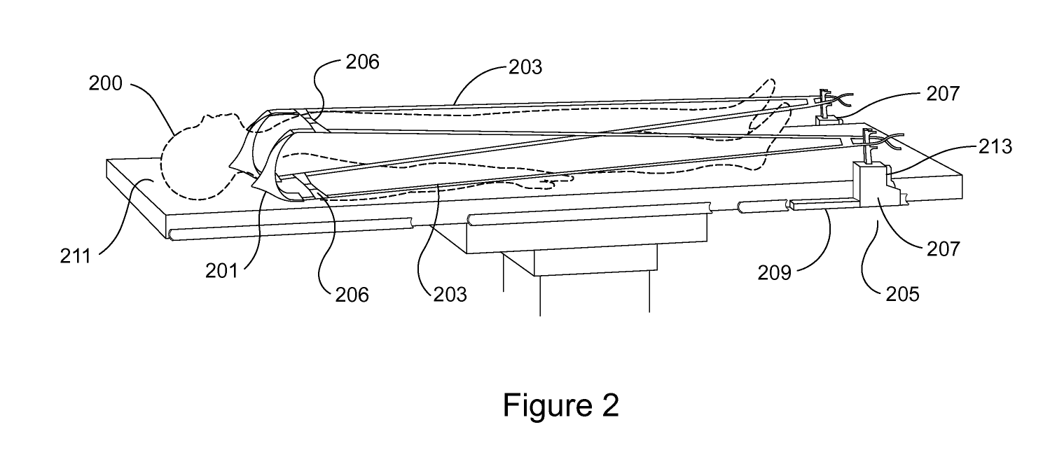

FIG. 2 is a side perspective view of an exemplary shoulder positioning system for improved lateral imaging of the cervical vertebral structure of a subject 200, in accordance with an embodiment of the present invention. In the present embodiment, arches 201 connect to straps 203 for distal migration of the shoulders of subject 200 via actuation of a table mounted locking system 205. Straps 203 may be connected to arches 201 by various different means for example, without limitation removable attachment means including, but not limited to, snaps, buckles, hook and loop material, etc. or permanent attachment means such as, but not limited to, adhesives or sewing. In some embodiments the arches may comprise various different features such as, but not limited to, holes, slots, or channels into which straps may be inserted. In the present embodiment, arches 201 are preferably made of a rigid radiolucent material so as not to block the view of the cervical vertebrae when obtaining a lateral image. Some non-limiting examples of rigid radiolucent materials that may be used to construct arches 201 include, without limitation, carbon, carbon fiber, PEEK, berylium, glass fiber, reinforced acrylics, thermoplastics, polycarbonates, polyketones, etc. In some embodiments, arches 201 may be padded for the comfort of subject 200. Straps 203 may be adjustable in length via various different adjustment means such as, but not limited to, sliding buckles, conventional buckles, ratcheting clasps, hooks and eyes, hook and loop material, etc. In alternate embodiments the straps may not be adjustable. Rather, in these embodiments, different sizes of straps may be made available for subjects of different heights. In the present embodiment, straps 203 are made of a durable material such as, but not limited to, heavy duty fabric, Kevlar®, polypropelene, braided nylon cord, cotton canvas/cotton duck, plastics, vinyl fabric & vinyl composites, vinyl coated mesh, Luma®-Fab GT, engineered polymer, stainless steel electromagnetic shielding textile, composite ripstop, denier nylon, lycra & lycra blend, polyurethane laminate, Textiline®, mylar, Canvak® waxed canvas, ballistic & non-ballistic Cordura®, Sorbtex® Poly Mesh, neoprene, hypalon, Toughtek® Leather, braided rope, In the present embodiment straps 203 are configured for, but not limited to, repeated usage . In alternate embodiments, straps of varying sizes, configurations and closures may be fashioned for one time disposable usage.

In the present embodiment, straps 203 connect arches 201 to table mounted locking system 205. It is contemplated that in alternate embodiments items other than straps may be used to connect the arches to the table mounted locking system such as, but not limited to, rope, cable, tubing, rigid rods, flexible rods, etc. In the present embodiment, adjustable cross straps 206 connect straps 203 across the chest and under the shoulder blades of subject 200 in order to generally prevent lateral migration of arches 201 from proper positioning over the acromionclavicular joint. Those skilled in the art, in light of the present teachings, will readily recognize that cross straps 206 may be made adjustable using various different means. For example, without limitation, cross straps 206 may comprise adjustment means such as, but not limited to, buckles or hook and loop material, or cross straps 206 may comprise a multiplicity of attachment points such as, but not limited to, snaps at which cross straps may be attached to straps 203. Cross straps 206 may be permanently attached to straps 203 or may be removable. Alternate embodiments may be implemented without such cross straps, with various different combinations of cross straps or with harnesses rather than cross straps to generally ensure non-migration of the arches.

In the present embodiment, table mounted braking system 205 comprises two rail guided unidirectional braking mechanisms 207 on two guide rails 209 mounted to opposite sides of the distal end of a surgical table 211. Straps 203 may be connected to braking mechanisms 207 using various different attachment means such as, but not limited to, clamps, loops or holes in straps 203, the tying of straps 203 to braking mechanisms 207, hooks, hook and loop, buttons, fitment via a rigid receptacle over a dedicated attachment point, threading and securement thru rigid attachment point via buckles, snaps, slides, zippers, Velcro®, etc. Unidirectional braking mechanisms 207 pull on straps 203 distally as opposed to pushing distally as shown in FIG. 1. Triggers 213 enable braking mechanisms 207 to be locked into place on guide rails 209 or to be released from guide rails 209. In alternate embodiments, brake mechanisms 207 may further comprise force sensors and displays for monitoring the amount of force being applied to the patient. In the present embodiment the following mechanism describes an exemplary braking mechanism without limitation to said mechanism. The exemplary braking mechanism comprises, but not limited to, a toothed rack fitted within the rail guide assembly, such that bidirectional free travel is permitted while the operator grips the spring loaded trigger actuator mechanism, thereby lifting and holding the locking pin above the toothed rack assembly. Brake actuation is affected via release of said trigger by the operator, thereby allowing the metallic, or non metallic, spring to force the locking pin into immediate rigid interface with the toothed rack assembly, restricting all motion, until such time as trigger actuation via the operator effects release of said rigid interface thru the lifting of the locking pin from the toothed rack assembly, thereby restoring free motion. In alternative embodiments, various friction and caliper braking mechanisms may be employed along the lines of bicycle or motorcycle disc or drum or caliper brakes arranged so as to replicate the above braking function with equivalent trigger actuation, as well as braking mechanisms which operate upon various mechanical means well known to one skilled in the art, to include, but not be limited to mechanical cone brakes, hydraulic braking systems, hydraulic clutches, mechanical clutches, pneumatic brakes, pneumatic clutches, friction/disc clutches, spring clutches, sprag clutches, roller ramp, electromagnetic clutches, gear drives, chain drives, etc. The mechanical means may involve, yet not be limited to, friction, wrap spring, oil shear, toothed surface, as well as non contact methods.

When triggers 213 are held down by a user, braking mechanisms 207 are able to slide along guide rails 209, and when triggers 213 are released, the brakes are engaged and braking mechanisms 207 are held securely in place on guide rails 209. Locking system 205 allows for the hands free usage of the positioning system during radiography, with quick release of all distal migratory tension to the shoulders of subject 200 via the simple tapping of triggers 213. Transmitting the distal migratory force to the shoulders through straps 203 enables locking system 205 to be placed at the foot of table 211. Therefore, locking system 205 does not necessarily need to enter the borehole of the imaging array and is not required to be low profile. Additionally, this placement positions high attenuation value components of the present invention out of the imaging array for purposes of elimination of potential sources of artifacts. Those skilled in the art, in light of the present teachings, will readily recognize that a multiplicity of alternate and suitable types of locking systems may be used in alternate embodiments such as, but not limited to, a ratcheting rack and pinion with a crank, various types of clamps, pins, etc. In the present exemplary embodiment automatic trigger actuation of the braking system is effected as soon as the operator releases said triggers as previously described. In alternative embodiments, the braking mechanism may be actuated via active means, such that activation is not automatic, but rather, requires the intentional activation of a lever, a knob, a set screw, a pin, a dial, a trigger, a switch, a clutch, etc. Furthermore, alternate embodiments may comprise various different means for variable strap positioning other than sliding braking mechanisms such as, but not limited to, spools, pulleys, etc. In the present embodiment, braking mechanisms 207 and guide rails 209 are preferably made of non fero-magnetic substances such as, but not limited to, aluminum , Delryn®, stainless steel and or titanium, which tough essentially radio-opaque, are nonetheless compatible with MRI/CT and Cone Beam Computed Tomagraphy. However, various different radiolucent and non radiolucent materials may be used such as, but not limited to, PEEK, carbon, thermoplastic resins, polypropylene, polyethylene, polyamides, polyphenylene sulfides, high performance polymers, polyaryletherketones, and carbon fiber reinforced thermoplastics. These materials may be extruded, compression molded, injection molded, or formed by other means.

In typical use of the present embodiment, after mounting braking mechanisms 207 to guide rails 209, subject 200 is placed on table 211. Straps 203, with arches 201 attached are then attached to braking mechanisms 207, and arches 201 are placed on the shoulders of subject 200 directly at the acromionclavicular joint. After cross straps 206 are in place, a user may effectively migrate the shoulders of subject 200 such that 12 to 14 pounds of distal migratory pressure, which is clinically proven as an effective amount of pressure for this application, is applied temporarily to the acromionclavicular joint by squeezing triggers 213 and pulling distally on braking mechanisms 207 as opposed to pushing distally as shown in FIG. 1. The application of distal motive force on braking mechanisms 207 by the user along with the hand actuation of triggers 213 moves arches 201 distally. Once the shoulders are properly migrated, the user releases triggers 213 to engage locking mechanisms 207 and locks the system into place. A clear lateral image of the cervical vertebral structures may then be obtained. Triggers 213 also enable a user to quickly and easily adjust the amount of pressure exerted by the system or to release subject 200 from the positioning system as needed during the procedure for various reasons such as, but not limited to, emergency situations or to give subject 200 a break. Some embodiments may comprise a quick release mechanism for the braking mechanisms so that the system may be immediately removed from the subject in case of an emergency. Once the imaging is complete, subject 200 may be released from the system. This means that subject 200 is only subjected to the distal migratory pressure during the lateral imaging, which in some applications may be as little as 1 to 2 minutes.

In the present embodiment, the positioning system works with the anatomy of subject 200 in order to provide migration of the shoulder, without migrating the entire body of subject 200, by pulling arches 201 from the foot of table 211 with straps 203. The acromionclavicular joint is designed by nature to migrate distally. With proper usage of this system, arches 201 make direct contact with this joint and transfer all distal motive energy directly via rigid positioning as opposed to straps that merely compress soft tissue or harnesses that pull the entire body or compress the trapezius muscle, which is not the source of a typical obscuring artifact during lateral radiography of the cervical vertebral structures. Cross straps 206 enhance this distal migration in the present embodiment by generally preventing lateral migration of arches 201 from the proper positioning directly over the acromionclavicular joint.

The present embodiment provides variable and rigid radiolucent positioning of the shoulder where prior art means of shoulder migration utilizing straps are not variably adjustable. These prior art means provide fixed positioning via weights hung from the table or via crude friction locks. Additionally, other prior art strap-actuated means pull at the wrist with the commonplace result of insult or injury to the brachial plexus nerve or to the wrists themselves via pulling on the soft tissue. Other prior art strap-activated means pull the entire trapezius muscle and shoulder simultaneously with the common effect of migrating the entire subject, either by lifting the subject upwards with the waist as a pivot point or via distal migration of the entire subject. The present embodiment delivers more effective and focused migration via placing rigid laminar arches 201 directly in contact with the bones of the acromion clavicular joint as opposed to pulling and compressing soft tissue. The present embodiment is also compatible with current imaging technology including, but not limited to, O-Arm and MRI.

Alternate embodiments of the present invention may be implemented with a single braking mechanism at the foot of the table rather than a pair of braking mechanisms. For example, without limitation, one such embodiment comprises a guide rail with a braking mechanism at the center of the foot of the table. In this embodiment, both straps, which are each connected to an arch, are connected to the braking mechanism, and the braking mechanism functions similarly to the braking mechanisms of the preferred embodiment described in the foregoing. Other alternate embodiments may comprise two braking mechanisms with a single trigger. In one such embodiment, the two braking mechanisms are located on opposite sides of the foot of the table and are connected by a crossbar, on which a single trigger is located. This trigger simultaneously controls both braking mechanisms. Therefore, by holding down the trigger and pulling on the crossbar, both braking mechanisms along with the connected straps and arches move distally to migrate a subject's shoulders. In other alternate embodiments, it is contemplated that braking mechanisms may comprise motors to provide the pulling force. These motors may be controlled at the table or remotely. Other alternate embodiments may control motors using a computing device with inputs from force sensors. In other alternate embodiments, the computing device may be remote using a wireless connection to motors and sensors. In yet other alternate embodiments, the computing device may work in conjunction with the imaging apparatus to apply the force only when needed.

Those skilled in the art, in light of the present teachings, will readily recognize that alternate embodiments of the present invention may be implemented for the positioning of various different portions of a subject's anatomy. For example, without limitation, in one alternate embodiment, the table mounted braking system along with an arch can readily be adapted as a vertical positioner for the leg for purposes such as, but not limited to, hip arthroplasty. Vertical positioning of the lower extremities can be affected via proximal migration of a table mounted guide rail arranged with straps secured to an arch with disposable padding, in such a manner as to capture the back end of the thigh as it intersects with the natural bend of the knee. Proximal migration of the table mounted braking system has the effect of raising the leg into an upright position. A vertical stabilizer bar may be positioned in such a way as to rise perpendicularly to the guide rail so as to generally prevent lateral motion of the limb. The table mounted braking system locks automatically when released, thereby effecting the full spectrum of adjustability.

In another alternate embodiment, by mounting the guide rail transversely at the side of the table, the system, turned sideways, may have great utility as lateral hip restraints or lateral shoulder restraints for use during hip and scoliosis procedures. The arch portion in this embodiment can be readily adapted to capture the hip of a subject positioned on his side. There are currently a number of hip positioners which accomplish this type of lateral hip support, via adjustable means utilizing either a peg board placed under the subject with means to plug the lateral hip supports into the peg board via a peg or via simple friction locks. However, neither of these solutions have the necessary strength to accommodate a bariatric patient, nor do these means offer the ease of use and intra-operative variability that may be provided by this embodiment of the present invention.

Yet another alternate embodiment may be used as lateral positioners that may be used with one hand in order to position and lock a subject into place. This embodiment involves transverse placement of the table mounted braking system, slight modification of the arch and a simple vertical plate equipped with a disposable pad in order to greatly improve upon the performance and function of prior art lateral positioners, none of which offer intra-operative variability or repositioning. In some such embodiments, the arch may be eliminated in favor of a padded strap or harness.

Having fully described at least one embodiment of the present invention, other equivalent or alternative methods of providing distal migration of the shoulders according to the present invention will be apparent to those skilled in the art. The invention has been described above by way of illustration, and the specific embodiments disclosed are not intended to limit the invention to the particular forms disclosed. For example, the particular implementation of the positioning system may vary depending upon the particular type of purpose for which it is to be used. The positioning systems described in the foregoing were directed to imaging implementations; however, similar techniques are to provide systems for distally migrating the shoulders that are operated from the foot of the patient platform that may be used in various different applications such as, but not limited to, the immobilization of a subject with a suspected neck or back injury, traction, physical therapy, etc. Non-imaging implementations of the present invention are contemplated as within the scope of the present invention. The invention is thus to cover all modifications, equivalents, and alternatives falling within the spirit and scope of the following claims.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. An apparatus comprising:
a first anatomic positioner for migrating a first anatomic part of a patient on a patient platform, said first anatomic positioner comprising:
an arch structure being configured to be operable for engaging the patient's shoulder area;
a strap structure being joined to said arch structure such that said strap structure is operable to extend caudally along the patient's side; and
a locking system being configured to be operable for longitudinal movement along a side of the patient platform and for being locked at a position along said movement, said locking system being further configured for engaging said strap structure for pulling said arch structure to caudally migrate the first anatomic part when said arch structure is operabley engaged with said first anatomic part of the patient.

2. The apparatus as recited in claim 1, in which said arch is further operable for engaging the patient's acromioclavicular joint and caudally migrating the acromioclavicular joint.

3. The apparatus as recited in claim 1, in which said locking system is further configured to be operated by an operator at a foot of the patient platform.

4. The apparatus as recited in claim 1, in which said arch structure comprises a radiolucent material.

5. The apparatus as recited in claim 1, in which said arch structure is substantially rigid.

6. The apparatus as recited in claim 1, in which said strap structure comprises a radiolucent material.

7. The apparatus as recited in claim 1, in which said strap structure further comprises separate ends for joining to legs of said first arch structure.

8. The apparatus as recited in claim 1, in which said strap structure further comprises two straps for joining to legs of said first arch structure.

9. The apparatus as recited in claim 1, in which said locking system further comprises a trigger mechanism for locking at the position.

10. The apparatus as recited in claim 1, in which said arch structure is padded.

11. The apparatus as recited in claim 1, further comprising a second anatomic positioner for migrating a second anatomic part of a patient on a patient platform, said second anatomic positioner comprising a mirror image of said first anatomic positioner.

12. The apparatus as recited in claim 11, further comprising a chest strap being joined between said strap structures of said first anatomic positioner and said second anatomic positioner to mitigate lateral migration of said arch structures.

13. The apparatus as recited in claim 12, further comprising a shoulder blade strap being joined between said strap structures of said first anatomic positioner and said second anatomic positioner to further mitigate lateral migration of said arch structures.

14. An apparatus comprising:
a first anatomic positioner for migrating a first acromioclavicular joint of a patient on a patient platform, said first anatomic positioner comprising:
means for engaging the patient's first acromioclavicular joint;
means, being joined to said engaging means, for extending caudally along the patient's first side; and
means, being joined to said extending means, for longitudinal movement along a first side of the patient platform, for pulling said engaging means to caudally migrate the first acromioclavicular joint, and for locking at a position along said movement where the first acromioclavicular joint has been migrated; and
a second anatomic positioner for migrating a second acromioclavicular joint of a patient on a patient platform, said second anatomic positioner comprising:
means for engaging the patient's second acromioclavicular joint;
means, being joined to said engaging means, for extending caudally along the patient's second side; and
means, being joined to said extending means, for longitudinal movement along a second side of the patient platform, for pulling said engaging means to caudally migrate the second acromioclavicular joint, and for locking at a position along said movement where the second acromioclavicular joint has been migrated to provide a clear radiographic lateral imaging of the cervical vertebral structures of the patient.

15. The apparatus as recited in claim 14, further comprising means for mitigating lateral migration of said engaging means.

16. An apparatus comprising:
a first anatomic positioner for migrating a first acromioclavicular joint of a patient on a patient platform, said first anatomic positioner comprising:
a substantially rigid arch being configured to be operable for engaging the patient's first acromioclavicular joint, said rigid arch comprising a radiolucent material;
a strap structure being joined to legs of said rigid arch to extend caudally along the patient's first side, said strap structure comprising a radiolucent material; and
a locking system being joined to said strap structure, said locking system being configured to be operable for longitudinal movement along a first side of the patient platform for pulling said arch structure to caudally migrate the first acromioclavicular joint, said locking system further comprising a trigger mechanism for locking at a position along said movement where the first acromioclavicular joint has been migrated; and
a second anatomic positioner for migrating a second acromioclavicular joint of a patient on a patient platform, said second anatomic positioner comprising:
a substantially rigid arch being configured to be operable for engaging the patient's second acromioclavicular joint, said rigid arch comprising a radiolucent material;
a strap structure being joined to legs of said rigid arch to extend caudally along the patient's second side, said strap structure comprising a radiolucent material; and
a locking system being joined to said strap structure, said locking system being configured to be operable for longitudinal movement along a second side of the patient platform for pulling said arch structure to caudally migrate the second acromioclavicular joint, said locking system further comprising a trigger mechanism for locking at a position along said movement where the second acromioclavicular joint has been migrated to provide a clear radiographic lateral imaging of the cervical vertebral structures of the patient.

17. The apparatus as recited in claim 16, further comprising a chest strap being joined between said strap structures to mitigate lateral migration of said arch structures.

18. The apparatus as recited in claim 17, further comprising a shoulder blade strap being joined between said strap structures to further mitigate lateral migration of said arch structures.

19. The apparatus as recited in claim 16, in which said locking systems are further configured to be operated by an operator at a foot of the patient platform.

20. The apparatus as recited in claim 16, in which said strap structures each further comprises two straps for joining to said legs of said arch structures.

21. The apparatus as recited in claim 16, in which said arch structures are padded.

22. A method of using the apparatus of claim 1, the method comprising steps of:
joining said locking system of said first anatomic positioner to the side of the patient platform proximate the foot of the patient platform where said locking system is operable to move longitudinally along the side;
joining said strap structure, joined to said arch structure, to said locking system;
placing said arch structure on the shoulder area of the patient;
pulling on said locking system to caudally migrate the first anatomic part; and
locking said locking system at the position when the first anatomic part has been migrated.

23. The method as recited in claim 22, further comprising the step of placing said arch structure on the shoulder area of the patient above the acromioclavicular joint.

24. The method as recited in claim 22, further comprising the step of operating said locking system from the foot of the patient platform.

25. The method as recited in claim 22, further comprising the step of operating a trigger mechanism for locking the locking system.

26. The method as recited in claim 22, further comprising steps of:
joining a locking system of a second anatomic positioner to a second side of the patient platform proximate the foot of the patient platform where said locking system is operable to move longitudinally along the second side;
joining a strap structure of said second anatomic positioner, joined to an arch structure of said second anatomic positioner, to said locking system;
placing said arch structure of said second anatomic positioner on a second shoulder area of the patient;
pulling on said locking system of said second anatomic positioner to caudally migrate a second anatomic part; and
locking said locking system of said second anatomic positioner at a position when the second anatomic part has been migrated.

27. The method as recited in claim 26, further comprising the step of placing said arch structure of said second anatomic positioner on the second shoulder area of the patient above a second acromioclavicular joint.

28. The method as recited in claim 26, further comprising the step of operating said locking system of said second anatomic positioner from the foot of the patient platform.

29. The method as recited in claim 26, further comprising the step of operating a trigger mechanism of said second anatomic positioner for locking the locking system of said second anatomic positioner.

30. The method as recited in claim 26, further comprising the step of joining a chest strap, on top of the patient's chest, between said strap structures of said first anatomic positioner and said second anatomic positioner to mitigate lateral migration of said arch structures.

31. The method as recited in claim 30, further comprising the step of joining a shoulder blade strap, beneath the patient's shoulder blades, between said strap structures of said first anatomic positioner and said second anatomic positioner to further mitigate lateral migration of said arch structures.
